(19) United States
(12) Patent Application Publication
(10) Pub. No.: US 2019/0059729 A1
(43) Pub. Date: Feb. 28, 2019

(54) SIGNALING DEVICE FOR CATHETERING REQUIREMENT

(71) Applicant: Volker Perlitz, Aachen (DE)

(72) Inventor: Volker Perlitz, Aachen (DE)

(21) Appl. No.: 16/112,699

(22) Filed: Aug. 25, 2018

(30) Foreign Application Priority Data
Aug. 25, 2017 (EP) 17187957.0

(51) Int. Cl. A61B 5/00; A61B 5/11; A61B 5/0205; G16H 10/40; A61B 5/087

(52) U.S. Cl. CPC A61B 5/0015; A61B 5/11; A61B 5/0205; A61M 25/0017; G16H 10/40; A61B 5/087; A61B 5/6898; A61B 5/7271

(57) ABSTRACT

A signaling device including a data processing device; sensors for heart frequency, breathing and movement of a person which respectively include radio communication devices for a wireless connection with the data processing device, wherein the data processing device is configured to wirelessly receive data captured by the sensors regarding a physiological condition of the person and to generate an acoustic, visual or tactile signal as a function of a change of the physiological condition, wherein the data processing device is configured to store the captured data in measurement series, to analyze the measurement series and to detect an increasing or leaping change of the condition in the measurement series, wherein the signal indicates a cathetering requirement of the person.

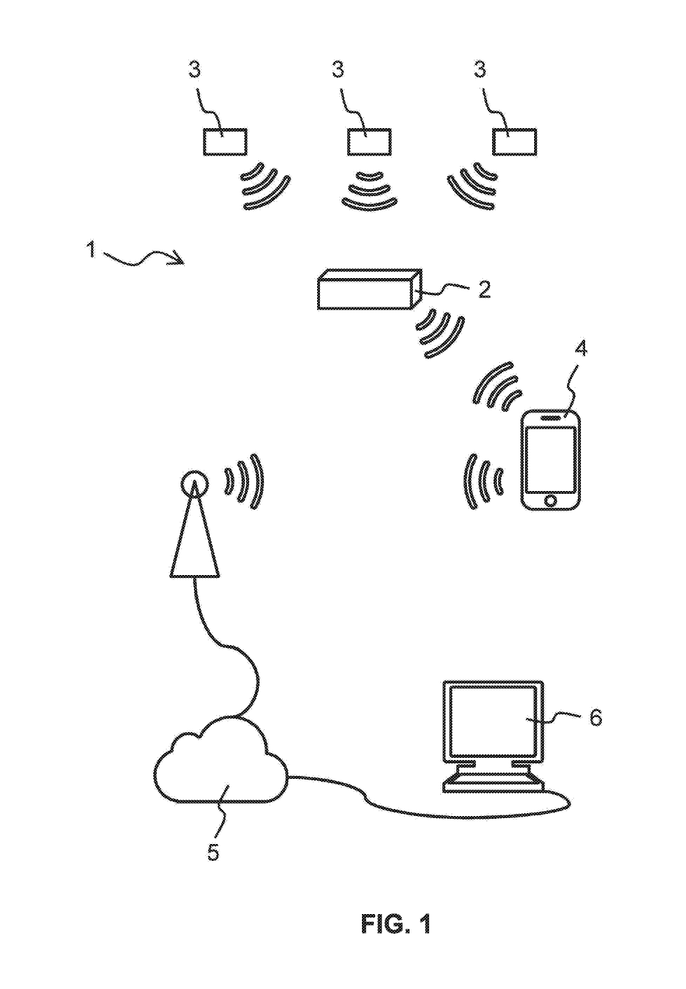

### RELATED APPLICATIONS

This application claims priority from and incorporates by reference European Patent Application 17 187 957.0 filed on Aug. 25, 2017.

### FIELD OF THE INVENTION

The invention relates to a signaling device for a cathetering requirement.

### BACKGROUND OF THE INVENTION

Pain from high bladder pressure is not directly perceived by persons with neurological damage (e.g., with paraplegia, multiple sclerosis or half-side paraplegia after apoplectic insult). A pain-stress stimulus of this type occurs individually in different manners in addition to motoric unrest with vegetative reactions like increased blood pressure, increased heart rate, breathing frequency, skin blood circulation, and electric skin conductivity.

In addition to treatment with medicines like anticholinergica and surgical measures like cutting the musculus sphincter vesical externus (Reynard J M, Vass J, Sullivan M E, Mamas M: Sphincterotomy and the treatment of detrusor-sphincter dyssenergia: current status, future prospects. Spinal Cord (2003) 41, 1-11, doi: 10.1038/sj.sc 3101378), intermittent cathetering is used to prevent an overfilling of the bladder, thus the bladder is emptied through a bladder catheter in approximately even intervals of approximately 6 hours.

Since neither urine production nor bladder capacity are constant, for example, infections or psychophysical stresses can reduce capacity, typically an overservicing occurs when the bladder is not yet filled sufficiently, or an underservicing occurs when it is already filled too much. When underservicing occurs, there can be secondary damages including kidney failure. Overservicing has to be prevented too because any insertion of a catheter being an invasive process always bears the risk of bacterial introduction with infection or an injury of the urinary tract (Frankel H L et al.: Long-term survival in spinal cord injury: a fifty year investigation. Spinal Cord 1988; 36: 868-869).

In order to detect an individual cathetering requirement before a high bladder pressure occurs, it is well known to estimate a filling condition of the bladder in a non-invasive manner by sonographic or impedance volumetric measurements (Schlebusch T: Impedanz-Zystovolumetrie. Aachen, Tech. Hochsch., Diss., 2015).

### BRIEF SUMMARY OF THE INVENTION

Thus it is an object of the invention to determine a suitable point in time for cathetering before excessive bladder pressure is generated.

According to the invention, a signaling device is proposed for a cathetering requirement, the signaling device including a data processing device and sensors for heart frequency, breathing and movement of a person, which respectively include radio transmission devices for wireless connection with the data processing device wherein the data processing device is configured to receive data received by the sensors regarding a physiological condition of the person wirelessly and to generate an acoustic, visual and/or tactile signal for an increasing (progressive) and/or excursive change of the condition. The signaling device according to the invention is configured to detect the cathetering requirement of a urine bladder of a person and to generate a signal for cathetering.

A screenless microcomputer is suitable in particular as a data processing device, typically including a proprietary voltage supply wherein the microcomputer is attached at a bed or a wheelchair or can be carried in a bag. Alternatively also a smartphone of the person is suitable as a data processing device.

In order to measure the heart frequency in particular conventional EKG sensors are suitable. For measuring breathing sensor mats or motion sensors are used, and for measuring subconscious movements of a body and/or also of limbs that are affected by paraplegia, motion sensors are used as well.

The radio connection of each individual sensor to the data processing device facilitates one the one hand side a free positioning of the sensors at the person or in the person's clothes, on the other hand side failure prone or bothersome cable connections are avoided and eventually the possibilities that are provided as a standard in microcomputers and mobile devices for radio connections, in particular Bluetooth, DECT and NFC can be used.

In the signal device according to the invention, the data processing device can be configured in particular through a software to wirelessly receive sensor-received data regarding a condition of the person and to generate an acoustic or tactile signal for an increasing (progressive) and/or leaping change of condition. The software device facilitates an adaptation of the data processing device to individually different or changing requirements and sensor configurations, but also in case of a malfunction, maintenance and repair, in particular by updating the software.

Detecting an increasing (progressive) and/or leaping change causes at least the preliminary storage of measurement values and knowledge of a time differential from the next measurement values.

The invention is based on the finding that the vegetative pain-stress reactions under high blood pressure are individually different for each patient but always connected with an increasing (progressive) and/or rapid change of breathing, skin blood circulation, subconscious movements, skin humidity or a combination of these features and which are furthermore essentially identical for each patient when repeated. The signaling device according to the invention facilitates an individual detection of the most important vegetative pain-stress reactions and thus a reliable signaling of a cathetering requirement when a high bladder pressure occurs.

Advantageously a signaling device according to the invention includes adjustment devices for manually adjusting a sensitivity of the sensors. The signaling device facilitates an individual adaptation to differently pronounced features of the pain-stress reaction.

Advantageously a signaling device according to the invention includes a control device for manually validating the signal. Thus, the signaling device facilitates a simple documentation and based thereon an adjustment of a threshold for detecting the increasing (progressive) and/or leaping condition change.

For adjustment and control devices on the one hand side mechanical twist and slide controls and keying devices can be used which can be configured in particular at the data processing device, alternatively virtual keys of such elements can be represented on a screen which is integrated into the data processing device and connected therewith, advantageously via radio, e.g., integrated in a smartphone.

Advantageously a signaling device of this type according to the invention includes an expert system for automatically calibrating a threshold value for the increasing (progressive) and/or leaping change based on manual validation of signals through the control device. Thus, the signaling device forms a continuously learning and self-adjusting system with reference to the threshold for detecting the increasing (progressive) and/or leaping condition change.

Advantageously a signaling device according to the invention includes additional sensors also for skin blood circulation and skin humidity, for the heart frequency and/or for the electrical activity of the brain of the person. The signaling device then includes further information regarding the condition of the person which can be relevant for detecting the increasing (progressive) and/or leaping condition change.

In order to measure skin blood circulation and arterial oxygen saturation, in particular sensors for reflection or transmission photo plethysmography (PPG, also pulse oximetry) are used, for measuring skin humidity sensors for electro dermal activity (galvanic skin response, GSR) are used, and for measuring the electric brain activity conventional EEG-sensors are used.

Advantageously a signaling device according to the invention includes a real-time clock. In the signal device the real time clock does not only provide a cyclic timing that is usable for determining a time differential from the preceding measurements but additionally also provides the option to document the measurements with absolute timestamps.

Advantageously a signaling device according to the invention includes a signaling element for generating the signal wherein the signaling element is wirelessly connected with the data processing device, e.g., integrated in a smartphone. Alternatively, the signaling device according to the invention can access a signaling element that is integrated in the data processing device. In particular vibrating elements, illuminants, as well as screens and speaker can be used as the signaling element.

Advantageously a signaling device according to the invention includes a control element that is connected with the data processing device. The data processing device of the signaling device can be configured without control elements and can be arranged at a location that is safe from unauthorized or unintentional access and from other external influences. The control element is advantageously wirelessly connected with the data processing device. Alternatively also a cable-based solution is suitable.

Advantageously the control element in a signaling device according to the invention is implemented in a smartphone. For example, the control element can be a software application or a website that is provided by the data processing device wherein the website is called up in a browser of the smartphone. Alternatively, the control element can also be implemented as a software application or a website on a personal computer (PC) that is only temporarily connected with the data processing device by cable. Alternatively the control element can also be a server application on a server of the manufacturer of the signaling device wherein the data processing device connects self-acting with the server or upon request through a GSM-module or through WLAN.

### BRIEF DESCRIPTION OF THE DRAWING

The invention is illustrated based on an embodiment with respect to a drawing FIGURE that illustrates the signaling device 1 according to the invention.

### DETAILED DESCRIPTION OF THE INVENTION

The signaling device 1 illustrated in the drawing FIGURE includes a data processing device 2, three sensors 3, and a user application that is installed on a smartphone 4 but not illustrated in more detail.

The sensors 3 are configured as a sensor mat for measuring electrical heart activity (EKG) and breathing of a person that is not illustrated, a reflection photo plethysmography sensor for measuring skin blood circulation, a motion sensor for measuring voluntary and involuntary movements and a GSR sensor for measuring skin humidity. The sensors 3 respectively include a Bluetooth radio module.

The data processing device 2 is a commercially available microcomputer including a processing, an operating memory, a real time clock and a Bluetooth radio module which is configured with a software to wirelessly receive data regarding a condition of a person that is captured by the sensors 3 and to generate a signal for an increasing (progressive) and/or leaping condition change.

In order to use the signaling device 1 the sensors 3 are initially applied to locations at a body of the person which do not further limit a mobility of the typically physically handicapped person. Since pain-stress reactions of a neurologically impaired person do not always evenly occur in the body, the sensors 3 are applied at predilection locations that impair as little as possible. Then the data processing device 2 is connected with the smartphone 4 through Bluetooth and configured by the user application.

The data processing device 2 detects the sensors 3 arranged in the proximity, establishes a data connection with the sensors via Bluetooth and performs a start configuration of the measurement. The data processing device 2 thus defines a measurement rhythm, divides the distance between two measurements in time windows and assigns one of the time windows to each of the sensors 3. From this point in time forward, the sensors 3 transmit their respective measuring value regarding the condition of the person in the assigned time window to the data processing device 2 in the measuring rhythm.

The data processing device 2 analyzes the time series of the measurements and detects an increasing (progressive) and/or leaping change of the condition of the person self-acting with a sensibility that is initially very high. As soon as the data processing device 2 detects an increasing (progressive) and/or leaping change, the user application generates a tactile, acoustic and visual signal by vibration, a signal tone and an illumination of an LED indicating a cathetering requirement and asks the user to validate the cathetering requirement by actuating a key that is represented on the screen, thus to confirm or reject the cathetering requirement.

With each validated signal an expert system that is implemented in the data processing device 2 learns which increasing (progressive) and/or leaping change of the measured values indicates an actual cathetering requirement. A number of erroneous signals decreases approximately exponentially with the number of the signals. Analyzing the measurement series the data processing device 2 also determined the actually required measuring sensitivity of the measuring sensors 3 and the actually required measuring rhythms and adapts both possibly automatically.

Through the user application the user can increase or decrease the measuring sensitivity of the signaling device 1 overall and for each individual sensor any time through virtual slider elements and can lengthen or shorten the measuring rhythm.

The user application connects through the internet 5 with a server 6 of the manufacturer in regular intervals when the smartphone 4 provides a data connection and transmits anonymized operating data of the signaling device 1 to the server 6. Based on this data the user application as well the software of the data processing device 2 is improved continuously.

### BRIEF DESCRIPTION OF THE DRAWINGS

1 Signaling device

2 Data processing device

3 Sensor

4 Smartphone

5 Internet

6 Server

What is claimed is:

1. A signaling device comprising:
a data processing device;
sensors for heart frequency, breathing and movement of a person which respectively include radio communication devices for a wireless connection with the data processing device,
wherein the data processing device is configured to wirelessly receive data captured by the sensors regarding a physiological condition of the person and to generate an acoustic, visual or tactile signal as a function of a change of the physiological condition,
wherein the data processing device is configured to store the data in measurement series, to analyze the measurement series and to detect an increasing or leaping change of the physiological condition in the measurement series,
wherein the signal indicates a cathetering requirement of the person.

2. The signaling device according to claim 1, further comprising: adjustment devices for manually adjusting a sensitivity of the sensors.

3. The signaling device according to claim 1, further comprising: a control device for manually validating the signal.

4. The signaling device according to claim 3, further comprising: an expert system configured to automatically calibrate a threshold value for the increasing or leaping change based on a manual validation of the signal by the control device.

5. The signaling device according to claim 1, further comprising: additional sensors for skin blood circulation and skin humidity, for heart frequency or for electrical activity of a brain of the person.

6. The signaling device according to claim 1, further comprising: a real time clock.

7. The signaling device according to claim 1, further comprising: a signaling element that is wirelessly connected with the data processing device and configured to generate the signal.

8. The signaling device according to claim 1, further comprising: a control element that is connected with the data processing device.

9. The signaling device according to claim 1, wherein the control element is implemented in a smartphone.
